(19) United States
(12) Patent Application Publication
(10) Pub. No.: US 2025/0058104 A1
(43) Pub. Date: Feb. 20, 2025

(54) IMPLANTABLE MEDICAL SYSTEMS FOR CANCER TREATMENT WITH ASYMMETRIC CURRENT DISTRIBUTION AMONGST ELECTRODES

(71) Applicant: Boston Scientific Scimed, Inc., Maple Grove, MN (US)

(72) Inventors: Brian L. Schmidt, White Bear Lake, MN (US); Devon N. Arnholt, Shoreview, MN (US); Michael J. Lyden, Shoreview, MN (US); Michael J. Kane, St. Paul, MN (US); Tucker James Nelson, Bloomington, MN (US)

(73) Assignee: MAYO FOUNDATION FOR MEDICAL EDUCATION AND RESEARCH, Rochester, MN (US)

(21) Appl. No.: 18/799,398

(22) Filed: Aug. 9, 2024

Related U.S. Application Data
(60) Provisional application No. 63/532,803, filed on Aug. 15, 2023.

(51) Int. Cl. A61N 1/36; A61N 1/05; A61N 1/372; A61N 1/375

(52) U.S. Cl. CPC A61N 1/36002; A61N 1/0529; A61N 1/36125; A61N 1/36157; A61N 1/36171; A61N 1/36175; A61N 1/37217; A61N 1/3754; A61N 1/3756

(57) ABSTRACT

Embodiments herein relate to implantable systems for cancer treatment and related methods. In an embodiment, an implantable system for cancer treatment is included having a plurality of implantable stimulation leads, a plurality of electric field therapy electrodes, a therapy output circuit, and control circuitry. The therapy output circuit can generate an electrical current for the plurality of electric field therapy electrodes to create one or more electric fields that are effective to prevent and/or disrupt cellular mitosis in a cell. The control circuitry can be configured to cause the electrical current to be distributed asymmetrically between working electrodes and counter electrodes amongst the plurality of electric field therapy electrodes. At least some of the plurality of electric field therapy electrodes are disposed on the plurality of implantable stimulation leads and in electrical communication with the therapy output circuit. Other embodiments are also included herein.

This application claims the benefit of U.S. Provisional Application No. 63/532,803, filed Aug. 15, 2023, the content of which is herein incorporated by reference in its entirety.

### FIELD

Embodiments herein relate to implantable systems for cancer treatment and related methods. More specifically, embodiments herein relate to implantable systems for cancer treatment configured to distribute current asymmetrically amongst electrodes.

### BACKGROUND

According to the American Cancer Society, cancer accounts for nearly 25% of the deaths that occur in the United States each year. The current standard of care for cancerous tumors can include first-line therapies such as surgery, radiation therapy, and chemotherapy. Additional second-line therapies can include radioactive seeding, cryotherapy, hormone or biologics therapy, ablation, and the like.

Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Various cancer therapies may have significant side effects on heathy tissue. Such side effects can vary widely depending on the type of cancer therapy but can manifest as anemia, thrombocytopenia, edema, alopecia, infections, neutropenia, lymphedema, cognitive problems, nausea and vomiting, neuropathy, skin and nail changes, sleep problems, urinary and bladder problems, and the like.

### SUMMARY

Embodiments herein relate to implantable systems for cancer treatment and related methods. In a first aspect, an implantable system for cancer treatment is included having a plurality of implantable stimulation leads, a plurality of electric field therapy electrodes, and a therapy output circuit. The therapy output circuit can be configured to generate an electrical current for the plurality of electric field therapy electrodes to create one or more electric fields. Control circuitry can cause the therapy output circuit to generate the one or more electric fields at frequencies selected from a range of between 10 kHz to 1 MHz within a bodily tissue. The one or more electric fields can be effective to prevent and/or disrupt cellular mitosis in a cell. The control circuitry can be configured to cause the electrical current to be distributed asymmetrically between working electrodes and counter electrodes amongst the plurality of electric field therapy electrodes. At least some of the plurality of electric field therapy electrodes can be disposed on the plurality of implantable stimulation lead and in electrical communication with the therapy output circuit.

In a second aspect, in addition to one or more of the preceding or following aspects, or in the alternative to some aspects, the control circuitry can be configured to cause the electrical current to be distributed asymmetrically between working electrodes and counter electrodes amongst the plurality of electric field therapy electrodes by pairing unequal numbers of working electrodes with counter electrodes.

In a third aspect, in addition to one or more of the preceding or following aspects, or in the alternative to some aspects, the control circuitry can be configured to cause the electrical current to be distributed asymmetrically between working electrodes and counter electrodes amongst the plurality of electric field therapy electrodes by duty cycling different working electrodes with a particular counter electrode and/or different counter electrodes with a particular working electrode.

In a fourth aspect, in addition to one or more of the preceding or following aspects, or in the alternative to some aspects, the system can further include a metallic housing, wherein the therapy output circuit can be disposed within the metallic housing.

In a fifth aspect, in addition to one or more of the preceding or following aspects, or in the alternative to some aspects, the metallic housing can be configured to serve as a working electrode or a counter electrode.

In a sixth aspect, in addition to one or more of the preceding or following aspects, or in the alternative to some aspects, can further include a plurality of current monitors, wherein the plurality of current monitors can be configured to monitor current to the plurality of electric field therapy electrodes.

In a seventh aspect, in addition to one or more of the preceding or following aspects, or in the alternative to some aspects, can further include a plurality of variable resistors, wherein the plurality of variable resistors can be configured to change impedance for the plurality of electric field therapy electrodes.

In an eighth aspect, a method of providing cellular mitosis disrupting cancer therapy can be included. The method can include generating an electrical current for a plurality of electric field therapy electrodes to create one or more electric fields and causing the electrical current to be distributed asymmetrically between working electrodes and counter electrodes amongst the plurality of electric field therapy electrodes.

In a ninth aspect, in addition to one or more of the preceding or following aspects, or in the alternative to some aspects, the method can further include causing the electrical current to be distributed asymmetrically between working electrodes and counter electrodes amongst the plurality of electric field therapy electrodes by pairing unequal numbers of working electrodes with counter electrodes.

In a tenth aspect, in addition to one or more of the preceding or following aspects, or in the alternative to some aspects, the method can further include causing the electrical current to be distributed asymmetrically between working electrodes and counter electrodes amongst the plurality of electric field therapy electrodes by duty cycling different working electrodes with a particular counter electrode and/or different counter electrodes with a particular working electrode.

In an eleventh aspect, in addition to one or more of the preceding or following aspects, or in the alternative to some aspects, the method can further include a housing serving as a working electrode or a counter electrode.

In a twelfth aspect, in addition to one or more of the preceding or following aspects, or in the alternative to some aspects, the method can further include monitoring current to the plurality of electric field therapy electrodes.

In a thirteenth aspect, in addition to one or more of the preceding or following aspects, or in the alternative to some aspects, the method can further include changing impedance for the plurality of electric field therapy electrodes.

In a fourteenth aspect, an implantable system for cancer treatment can be included having a plurality of implantable stimulation lead, a plurality of electric field therapy electrodes, and a therapy output circuit. The therapy output circuit can be configured to generate an electrical current for the plurality of electric field therapy electrodes to create one or more electric fields. Control circuitry can cause the therapy output circuit to generate the one or more electric fields at frequencies selected from a range of between 10 kHz to 1 MHz within a bodily tissue, wherein the one or more electric fields can be effective to prevent and/or disrupt cellular mitosis in a cell. The control circuitry can be configured to cause the electrical current to be distributed between working electrodes and counter electrodes amongst the plurality of electric field therapy electrodes such that some electrodes have a higher operating temperature during therapy than other electrodes. At least some of the plurality of electric field therapy electrodes can be disposed on the plurality of implantable stimulation lead and in electrical communication with the therapy output circuit.

In a fifteenth aspect, in addition to one or more of the preceding or following aspects, or in the alternative to some aspects, the control circuitry can be configured to cause the electrical current to be distributed between working electrodes and counter electrodes amongst the plurality of electric field therapy electrodes such that some electrodes have a higher operating temperature or lower operating temperature during therapy than other electrodes by distributing current amongst the electrodes asymmetrically.

In a sixteenth aspect, in addition to one or more of the preceding or following aspects, or in the alternative to some aspects, the control circuitry can be configured to cause the electrical current to be distributed asymmetrically between working electrodes and counter electrodes amongst the plurality of electric field therapy electrodes by pairing unequal numbers of working electrodes with counter electrodes.

In a seventeenth aspect, in addition to one or more of the preceding or following aspects, or in the alternative to some aspects, the control circuitry can be configured to cause the electrical current to be distributed asymmetrically between working electrodes and counter electrodes amongst the plurality of electric field therapy electrodes by duty cycling different working electrodes with a particular counter electrode and/or different counter electrodes with a particular working electrode.

In an eighteenth aspect, in addition to one or more of the preceding or following aspects, or in the alternative to some aspects, the system can further include a metallic housing, wherein the therapy output circuit can be disposed within the metallic housing.

In a nineteenth aspect, in addition to one or more of the preceding or following aspects, or in the alternative to some aspects, the metallic housing can be configured to serve as a working electrode or a counter electrode.

In a twentieth aspect, in addition to one or more of the preceding or following aspects, or in the alternative to some aspects, can further include a plurality of current monitors, wherein the plurality of current monitors can be configured to monitor current to the plurality of electric field therapy electrodes.

In a twenty-first aspect, in addition to one or more of the preceding or following aspects, or in the alternative to some aspects, can further include a plurality of variable resistors, wherein the plurality of variable resistors can be configured to change impedance for the plurality of electric field therapy electrodes.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following drawings, in which:

FIG. 1 is a schematic view of a medical device implanted in a patient in accordance with various embodiments herein.

FIG. 2 is a schematic view of a placement of various electrical field therapy leads in accordance with various embodiments herein.

FIG. 3 is a schematic view of a placement of electrical field therapy leads with respect to a tumor in accordance with various embodiments herein.

FIG. 4 is a schematic view of electrodes and current distribution thereto with respect to a tumor in accordance with various embodiments herein.

FIG. 5 is a schematic view of electrodes and current distribution thereto with respect to a tumor in accordance with various embodiments herein.

FIG. 6 is a schematic view of a cancer therapy lead in accordance with various embodiments herein.

FIG. 7 is a schematic cross-sectional view of a cancer therapy lead in accordance with various embodiments herein.

FIG. 8 is a schematic cross-sectional view of a medical device in accordance with various embodiments herein.

FIG. 9 is a schematic diagram of components of a medical device in accordance with various embodiments herein.

While embodiments are susceptible to various modifications and alternative forms, specifics thereof have been shown by way of example and drawings, and will be described in detail. It should be understood, however, that the scope herein is not limited to the particular embodiments described. On the contrary, the intention is to cover modifications, equivalents, and alternatives falling within the spirit and scope herein.

### DETAILED DESCRIPTION

Cancer therapies including the application of electrical fields may generate an amount of heat. For example, generation of electrical fields to prevent and/or disrupt cellular mitosis may generate heat. In some cases, the heat may be enough to raise the temperature of tissues to levels resulting in negative tissue effects and/or tissue damage. Issues with heat exposure can be particularly important in the context of treating tissue inside the head such as in the case of a brain tumor and/or the treatment of a tumor resection site.

In some embodiments of systems herein can use techniques of asymmetric current distribution in order to keep some electrodes cooler than others. For example, if a particular electrode is close to particularly sensitive tissue, then the system can keep that electrode cooler than others by using techniques of asymmetric current distribution. Conversely, if a particular electrode is in an area where thermal effects on tissue are not a concern (such as with an electrode disposed within a tumor), the system can allow such an electrode to be hotter than other electrodes.

Beyond managing heat associated with implanted electrodes, systems herein can also use techniques of asymmetric current distribution to address other scenarios including, but not limited to, electrodes with different surface areas (the larger the effective surface area, the smaller the impedance), adjusting for different tissue impedances, etc.

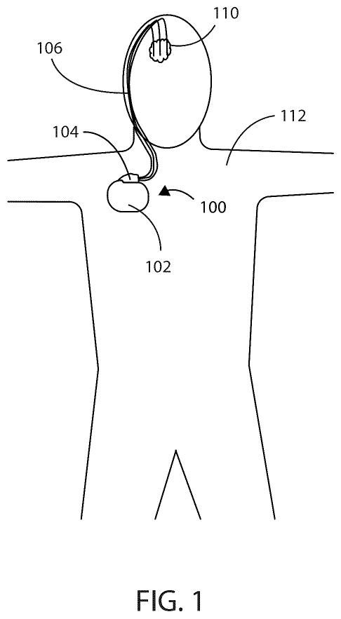

Referring now to FIG. 1, a schematic view of a medical device 100 implanted in a patient 112 is shown in accordance with the embodiments herein. In FIG. 1, the patient 112 has the medical device 100 implanted entirely within the body of the patient 112 at or near a tumor 110 or a tumor resection site. It will be the area at or near tumor 110 can also represent a tumor resection site, a cancerous or non-cancerous tumor site, zone, or cavity, a targeted treatment area, or the like. Various implant sites can be used including areas such as in the limbs, the upper torso, the abdominal area, the head, and the like. In some embodiments, the medical device can be at least partially implanted within the body of the patient at or near the site of the cancerous tumor, tumor resection site, etc.

The medical device 100 can include a housing 102 and a header 104 coupled to the housing 102. Various materials can be used. However, in some embodiments, the housing 102 can be formed of a material such as a metal, ceramic, polymer, composite, or the like. In some embodiments, the housing 102, or one or more portions thereof, can be formed of titanium. The header 104 can be formed of various materials, but in some embodiments the header 104 can be formed of a translucent polymer such as an epoxy material. In some embodiments the header 104 can be hollow. In other embodiments the header 104 can be filled with components and/or structural materials such as epoxy or another material such that it is non-hollow.

In some embodiments, such as where a portion of the medical device 100 is partially external, the header 104 and housing 102 can be surrounded by a protective casing made of durable polymeric material. In other embodiments, where a portion of the medical device 100 is partially external, the header 104 and housing 102 can be surrounded by a protective casing made of a combination of polymeric material, metallic material, and/or glass material.

The header 104 can be coupled to one or more leads, such as leads 106. The header 104 can serve to provide fixation of the proximal end of one or more leads 106 and electrically couple the one or more leads 106 to one or more components within the housing 102.

The one or more leads 106 can include one or more electrodes (not shown in this view) disposed along the length of the leads 106. In some embodiments, electrodes can include supply electrodes, also referred to herein as “electric field therapy supply electrodes.” Such supply electrodes can include working electrodes and counter electrodes. In some embodiments electrodes can include electric field sensing electrodes, also referred to herein as “sensing electrodes.” In some embodiments, leads 106 can include both supply electrodes and sensing electrodes. In other embodiments, leads 106 can include any number of electrodes that are both supply electrodes and sensing electrodes. In many embodiments, the electrodes described herein are implanted within a subject. However, in some embodiments, electrodes herein could also be non-implanted. For example, electrodes herein could include electrodes disposed on or over the skin.

The one or more leads 106 can also include one or more temperature sensors (not shown in this view) disposed along the length of the leads 106. Temperature sensors herein can include, but are not limited to, various types of optical and electrical temperature sensors. Temperature sensors herein can include contact-type temperature sensors and non-contact type temperature sensors. Optical temperature sensors herein can include infrared optical temperature sensors. Some optical temperature sensors can measure temperature at a distance such as a distance of millimeters or centimeters. Thus, even where temperature sensors are mounted along a lead 106, temperature can be measured at a distance therefrom. Exemplary electrical temperature sensors can include, but are not limited to, thermistors, resistive temperature detectors, thermocouples, semiconductor-based temperature sensors, and the like.

In some embodiments, the medical device system can include a temperature sensor disposed remotely from the medical device. A remote temperature sensor can provide temperature data in addition to or in replace of temperature sensors in other areas such as along the leads 106. In some embodiments, a remote temperature sensor can be used to gather a core or reference temperature of the patient into which the system is implanted.

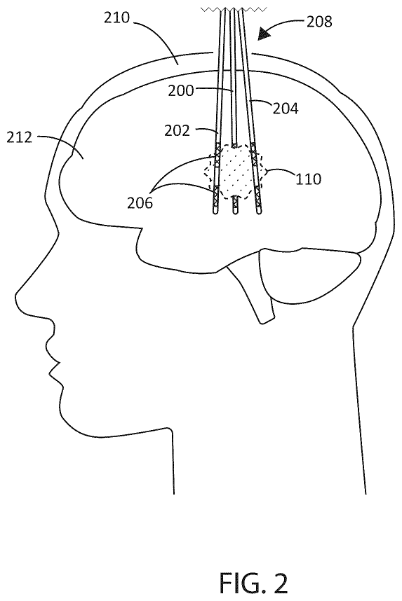

In some embodiments, the medical device can include a plurality of therapy leads implanted at or near a site a cancerous tumor or tumor resection. Referring now to FIG. 2, a schematic view of a placement of various cancer therapy leads 200, 202, 204 in a region of a tumor 110 or tumor resection site is shown in accordance with various embodiments herein. In the embodiment shown in FIG. 2, each cancer therapy lead includes two supply electrodes 206 disposed along a length of the cancer therapy leads. Each cancer therapy lead includes a proximal and a distal supply electrode. The cancer therapy leads and the supply electrodes disposed thereon are discussed in more detail below.

The side view shown in FIG. 2 also includes the placement of cancer therapy leads 200, 202, and 204 around the tumor 110 and in position within a burr hole 208 entry point on the patient's skull 210 within the patient's brain 212. It will be appreciated that in some embodiments one burr hole can be used with one or more (e.g., one, two, three, or more) leads and/or electrodes. In some embodiments, multiple burr holes can be used each with one or more (e.g., one, two, three, or more) leads and/or electrodes.

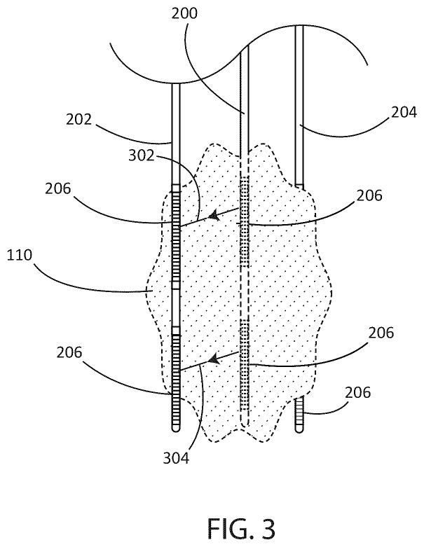

Referring now to FIG. 3, a schematic view of the placement of electrical field therapy leads with respect to a tumor is shown in accordance with various embodiments herein. FIG. 3 shows cancer therapy leads 200, 202, 204 in a region of a tumor 110 or tumor resection site (or targeted therapy site, etc.). In the embodiment shown in FIG. 2, each cancer therapy lead includes two supply electrodes 206 disposed along a length of the cancer therapy leads. Each cancer therapy lead includes a proximal and a distal supply electrode. It will be appreciated that therapy leads herein can include greater or lesser numbers of electrodes.

Electric fields can be generated along different vectors 302, 304 associated with therapy electrodes (such as working electrodes paired with counter electrodes). FIG. 3 depicts a 1 to 1 association of working electrodes with counter electrodes. However, systems herein can be configured to pair an unequal number of working electrodes and counter electrodes. For example, systems herein can be configured to distribute current to electrodes asymmetrically including working electrodes paired with counter electrodes in ratios other than 1:1. For example, while not particularly limited, such ratios can include 1:2, 1:3, 1:4, 1:5, 1:6, 2:1, 3:1, 4:1, 5:1, 6:1, 2:3, 2:4, 2:5, 2:6, 3:2, 4:2, 5:2, 6:2, 3:4, 3:5, 3:6, 4:3, 5:3, 6:3 (working electrodes paired with counter electrodes) and the like.

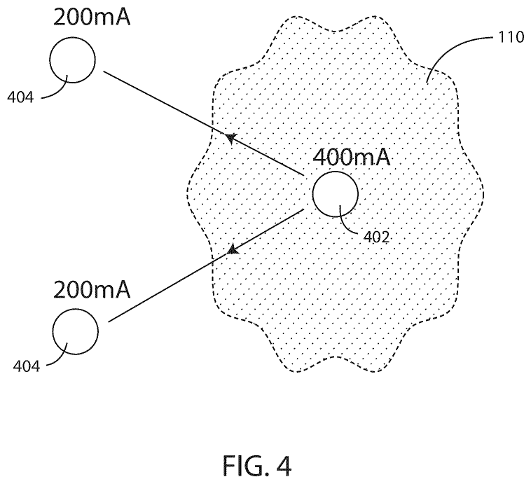

Referring now to FIG. 4, a schematic view of electrodes and current distribution thereto with respect to a tumor in accordance with various embodiments herein. In this example, the working electrode 402 can be paired with two counter electrodes 404. For sake of illustration, if the system provided 400 mA of current to working electrode 402, then the current distribution can effectively be split between counter electrodes 404 with each receiving 200 mA. In this case, the heat generated by the two counter electrodes 404 would be substantially less than the heat generated by the working electrode 402. It will be appreciated that the currents described with respect to FIG. 4 are just provided by way of example and that actual current amounts can vary substantially. Also, it will be appreciated that the current does not have to be evenly split with embodiments herein. For example, in some cases, 55, 60, 65, 70, 75, 80, 85, 90, 95, 98, 99 percent, or more of the current can pass through one counter electrode while the other counter electrode can receive the balance. In addition, splitting of current can occur across more than just two electrodes. For example, current can be split across 2, 3, 4, 5, 6, 7, 8, 9, 10 or more electrodes (counter electrodes or working electrodes). Such current can be split in various ways. In one approach, hardware components such as variable resistors, variable capacitors, and/or field effect transistors, and the like, can be used to control impedance of conductive paths associated with particular electrodes (working electrodes and/or counter electrodes). The system can actively control the impedance of such conductive paths allowing the system to change how current is distributed between electrodes.

In some cases, the system can accept user input regarding electrodes that must remain relatively cooler versus those that can operate at higher temperatures during therapy. Then the system can distribute current accordingly such that designated electrodes stay cooler during operation. For example, as shown in FIG. 4, the working electrode 402 is disposed within a tumor 110. The potential of thermal damage to a tumor is generally not a concern and therefore a working electrode 402 positioned in this manner can be allowed to reach higher temperatures. In contrast, the counter electrodes 404 are disposed outside of the tumor 110 and there may be an interest in keeping them cooler. However, there are many other scenarios where temperature may be less of a concern for one electrode versus another.

It will be appreciated that the positions of the working electrodes and counter electrodes can be reversed with respect to the tumor or another treatment site. Further, in some embodiments, multiple working electrodes can be paired with a single counter electrode.

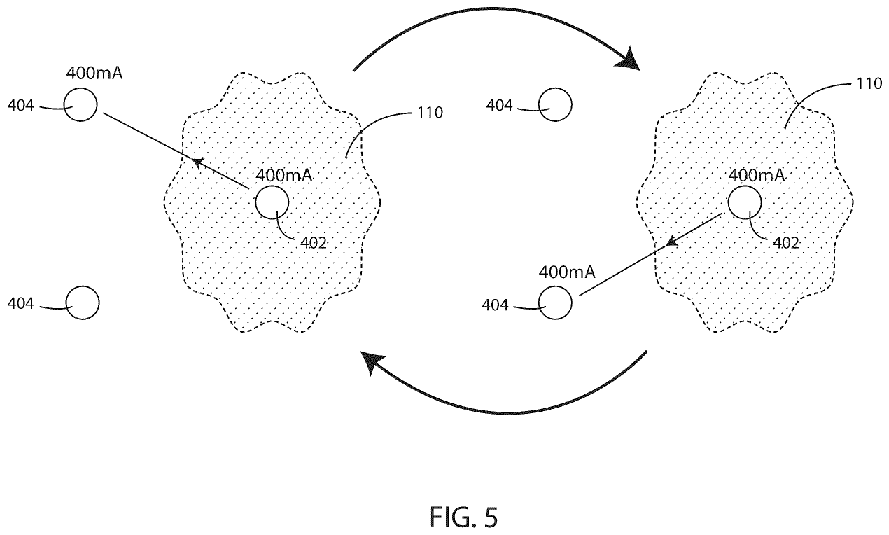

In some cases, another approach to current distribution involves duty cycling between electrodes. Referring now to FIG. 5, a schematic view of electrodes and current distribution thereto with respect to a tumor in accordance with various embodiments herein. In this example, the working electrode 402 can be paired with two counter electrodes 404. For sake of illustration, the system is shown providing 400 mA of current to working electrode 402. However, instead of splitting current between two counter electrodes in the manner illustrated with respect to FIG. 4, in this case duty cycling is used as shown in FIG. 5 such that the use of counter electrodes 404 alternates. In this manner, the time averaged magnitude of current for a given counter electrode 404 is substantially less than for the working electrode 402 resulting in substantially less heat being generated at the counter electrodes 404 than at the working electrode 402. The duty factor for each counter electrode 404 (also applicable to scenarios with multiple working electrodes and one counter electrode) can be controlled and can be evenly split at 50%, but could also vary, such as 90:10, 80:20, 70:30, 60:40, etc. In this manner, time averaged current, and therefore heat generated, can be controlled between multiple counter electrodes (and/or between multiple working electrodes). In some embodiments, the system can accept user input regarding the temperature sensitives of counter electrodes and/or working electrodes and distribute current to limit heat generation accordingly. Also, it will be appreciated that the duty cycle schema can include portions of time where the circuit is totally off and neither counter electrode is being utilized.

### Cancer Therapy Stimulation Leads

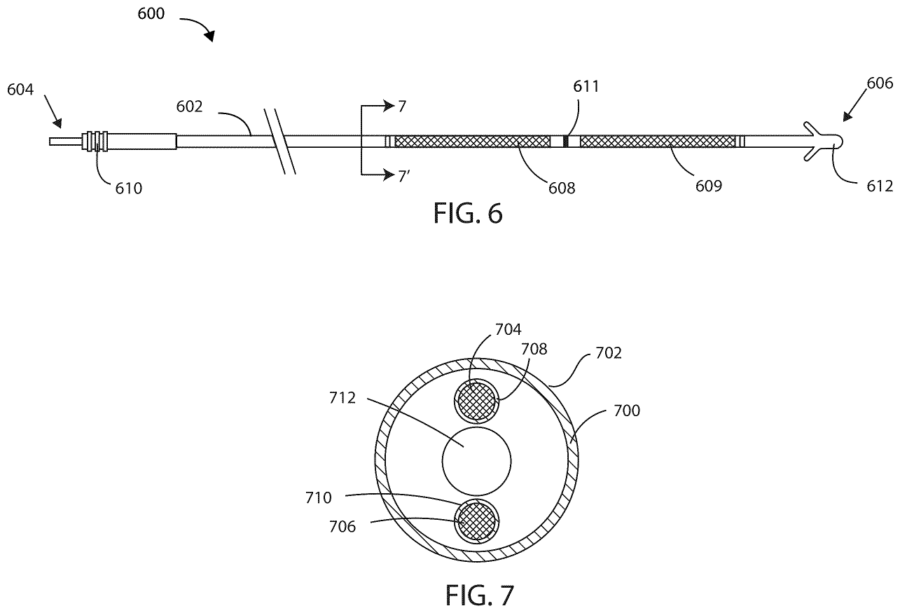

Referring now to FIG. 6, a schematic view of an exemplary cancer therapy stimulation lead 600 is shown in accordance with various embodiments herein. The cancer therapy stimulation lead 600 can include a lead body 602 with a proximal end 604 and a distal end 606. In this example, a first electrode 608 and a second electrode 609 are coupled to the lead body 602, as positioned near a distal end 606 thereof. In some embodiments, the electrodes 608, 609 can include electric field generating electrodes (which can function as working electrodes or counter electrodes depending on the system configuration). In various embodiments, the electrodes 608, 609 can include electric field sensing electrodes. The electrodes 608, 609 can be internally connected or internally independent. In an example where the electrodes 608, 609 are independent, the system herein can model each as an independent field and heat source. The lead body 602 can define a lumen. The electrodes 608, 609 can include various conductive materials such as platinum, silver, gold, iridium, titanium, and various alloys. In some embodiments, the cancer therapy stimulation lead 600 includes more than two electrodes.

The cancer therapy stimulation lead 600 can include one or more therapy zone temperature sensors disposed along a length of the cancer therapy stimulation lead. In this example, a therapy zone temperature sensor 611 is positioned between the first electrode 608 and the second electrode 609. However, the therapy zone temperature sensor 611 can also be positioned at various other points along or in the lead. In some embodiments, the therapy zone temperature sensor 611 can be positioned in the lead directly beneath an electrode. The therapy zone temperature sensor 611 can include an optical or electrical thermal sensor. For example, the therapy zone temperature sensor can include a thermistor. The therapy zone temperature sensor 611 can be used to measure the thermal heating about the cancer therapy stimulation lead to provide feedback to a clinician about the local thermal heating zone around the lead and provide a tissue temperature of the treatment site to the medical device.

In various embodiments, the therapy zone temperature sensor 611 can provide a tissue temperature at a site offset from a surface of the electrodes 608, 609. If a tissue temperature of a site offset from the electrodes 608, 609 is measured, the medical device can compensate for the offset when measuring or estimating the temperature of the tissue. In some embodiments, the therapy zone temperature sensor 611 can measure or estimate the reference or core body temperature of the patient when the therapy is turned off or paused. While not intending to be bound by theory, in some scenarios it can be easier to get an accurate measurement of a reference or core body temperature when therapy is turned off or paused. In some embodiments, therapy zone temperature sensor data can be recorded and relayed to the clinician, patient, care provider, and/or medical record system.

In some embodiments, systems herein can include temperature sensors individually associated with electrodes to allow the temperature of specific electrodes and/or tissues adjacent thereto to be assessed. In some embodiments, the system can dynamically adjust current distribution in order to prevent temperatures associated with specific electrodes and/or tissue adjacent thereto from exceeding temperature threshold values (preset, set through user input, calculated by the system, etc.). For example, if a particular electrode is sensed to approach or cross a threshold value for temperature, then the system can redistribute current (such as change how current is split or modifying duty cycle schemas) using techniques as described herein such that less heat is generated by the particular electrode.

The cancer therapy stimulation lead 600 can further include a terminal pin 610 for connecting the cancer therapy stimulation lead 600 to a medical device, such as a cancer treatment device. The terminal pin 610 can be compatible with various standards for lead-header interface design including the DF-1, VS-1, IS-1, LV-1 and IS-4 standards, amongst other standards.

In some embodiments, the cancer therapy stimulation lead 600 can further include a fixation element 612, such as an element that can adhere to a portion of the subject's body to maintain the position of the cancer therapy stimulation lead 600 and/or the electrodes 608. In various embodiments, the fixation element 612 can be disposed along the distal end 606 of the cancer therapy stimulation lead 600. However, in some embodiments a fixation element 612 is omitted.

Referring now to FIG. 7, a cross-sectional schematic view of a cancer therapy stimulation lead 600 as taken along line 7-7′ of FIG. 6 is shown in accordance with various embodiments herein. The cancer therapy stimulation lead 600 can include an outer layer 700 with an outer surface 702. The outer layer 700 can be flexible and can be configured to protect other components disposed within the lumen of the outer layer 700. In some embodiments, the outer layer 700 can be circular in cross-section. In some embodiments, the outer layer 700 includes a dielectric material and/or an insulator. In some embodiments, the outer layer 700 can include various biocompatible materials such as polysiloxanes, polyethylenes, polyamides, polyurethane and the like.

In various embodiments, the cancer therapy stimulation lead 600 can include one or more conductors, such as a first conductor 704 and a second conductor 706. In some embodiments, the first conductor 704 and the second conductor 706 can be disposed within the lumen of the outer layer 700. The first conductor 704 and a second conductor 706 can be configured to provide electrical communication between an electrode 608 and the proximal end 604 of the cancer therapy stimulation lead 600. The first conductor 704 and a second conductor 706 can include various materials including copper, aluminum, silver, gold, and various alloys such as tantalum/platinum, MP35N, stainless steel, and the like. An insulator 708 and 710 can surround the first conductor 704 and a second conductor 706. The insulators 708 and 710 can include various materials such as electrically insulating polymers.

In some embodiments, each of the electrodes 608 can have individual first conductors 704 and second conductors 706 to electrically couple the electrode 608 to the proximal end 604 of the cancer therapy stimulation lead 600. However, in some embodiments, each of the electrodes 608 only connects to a single conductor to electrically couple the electrode 608 to the proximal end 604 of the cancer therapy stimulation lead 600. In some embodiments, the first conductor 704 and a second conductor 706 can be configured as a coil or a cable. Multiple conductors can be disposed within the lumen of the outer layer 700. For example, a separate conductor or set of conductors can be in communication with each electrode disposed along the lead. In various embodiments, a first conductor 704 and a second conductor 706 can form a part of an electrical circuit by which the electric fields from the electric field generating circuit are delivered to the site of the cancerous tissue. Many more conductors than are shown in FIG. 7 can be included within embodiments herein. For example, the cancer therapy stimulation lead 600 can include 1, 2, 3, 4, 5, 6, 7, 8, 10, 15 or 20 or more conductors, or any number of conductors falling within a range between any of the foregoing.

In some embodiments, the cancer therapy stimulation lead 600 can include a central channel 712. The central channel 712 can be configured for a guide wire, or other implanting device, to pass through, such as to aid in implanting the cancer therapy stimulation lead 600 and electrodes 608. In some cases, additional channels (not shown) are disposed within the cancer therapy stimulation lead 600.

### Medical Device Components

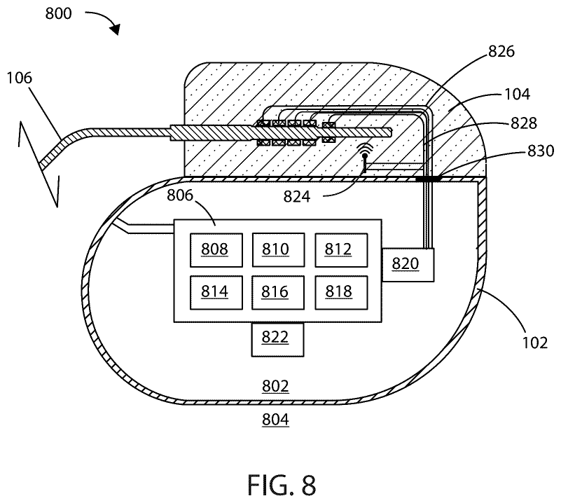

Referring now to FIG. 8, a schematic cross-sectional view of an exemplary medical device 800 is shown in accordance with various embodiments herein. The housing 102 can define an interior volume 802 that can be hollow and that in some embodiments is hermetically sealed off from the area 804 outside of medical device 800. In other embodiments the housing 102 can be filled with components and/or structural materials such that it is non-hollow. The medical device 800 can include control circuitry 806, which can include various components 808, 810, 812, 814, 816, and 818 disposed within housing 102. In some embodiments, these components can be integrated and in other embodiments these components can be separate. In yet other embodiments, there can be a combination of both integrated and separate components. The medical device 800 can also include an antenna 824, to allow for unidirectional or bidirectional wireless data communication, such as with an external device or an external power supply. In some embodiments, the components of medical device 800 can include an inductive energy receiver coil (not shown) communicatively coupled or attached thereto to facilitate transcutaneous recharging of the medical device via recharging circuitry.

The various components 808, 810, 812, 814, 816, and 818 of control circuitry 806 can include, but are not limited to, a microprocessor, memory circuit (such as random access memory (RAM) and/or read only memory (ROM)), recorder circuitry, controller circuit, a telemetry circuit, a power supply circuit (such as a battery), a timing circuit, and an application specific integrated circuit (ASIC), a recharging circuit, amongst others. Control circuitry 806 can be in communication with an electric field generating circuit 820 that can be configured to generate electric current to create one or more fields. The electric field generating circuit 820 can be integrated with the control circuitry 806 or can be a separate component from control circuitry 806. Control circuitry 806 can be configured to control delivery of electric current from the electric field generating circuit 820. In some embodiments, the electric field generating circuit 820 can be present in a portion of the medical device that is external to the body.

In some embodiments, the control circuitry 806 can be configured to direct the electric field generating circuit 820 to deliver an electric field via leads 106 to the site of a cancerous tumor located within a bodily tissue. In other embodiments, the control circuitry 806 can be configured to direct the electric field generating circuit 820 to deliver an electric field via the housing 102 of medical device 800 to the site of a cancerous tumor located within a bodily tissue. In other embodiments, the control circuitry 806 can be configured to direct the electric field generating circuit 820 to deliver an electric field between leads 106 and the housing 102 of medical device 800. In some embodiments, one or more leads 106 can be in electrical communication with the electric field generating circuit 820.

In some embodiments, medical device 800 can include an electric field sensing circuit 822 configured to generate a signal corresponding to sensed electric fields. Electric field sensing circuit 822 can be integrated with control circuitry 806 or it can be separate from control circuitry 806.

Sensing electrodes (not shown in this view) can be disposed on or adjacent to the housing of the medical device, on one or more leads connected to the housing, on a separate device implanted near or in the tumor, or any combination of these locations. In some embodiments, the electric field sensing circuit 822 can measure the electrical potential difference (voltage) between a first electrode and a second electrode, wherein the first and second electrodes are in any of the aforementioned locations. In some embodiments, the electric field sensing circuit can be configured to measure sensed electric fields and to record electric field strength in V/cm.

In some embodiments, the one or more leads 106 can be in electrical communication with the electric field generating circuit 820. The one or more leads 106 can include one or more electrodes. In some embodiments, various electrical conductors, such as electrical conductors 826 and 828, can pass from the header 104 through a feed-through structure 830 and into the interior volume 802 of medical device 800. As such, the electrical conductors 826 and 828 can serve to provide electrical communication between the one or more leads 106 and control circuitry 806 disposed within the interior volume 802 of the housing 102.

In some embodiments, recorder circuitry can be configured to record the data produced by the electric field sensing circuit 822 and record time stamps regarding the same. In some embodiments, the control circuitry 806 can be hardwired to execute various functions, while in other embodiments the control circuitry 806 can be directed to implement instructions executing on a microprocessor or other external computation device. A wireless communication interface can also be provided for communicating with external computation devices such as a programmer, a home-based unit, and/or a mobile unit (e.g., a cellular phone, personal computer, smart phone, tablet computer, smartwatch, and the like).

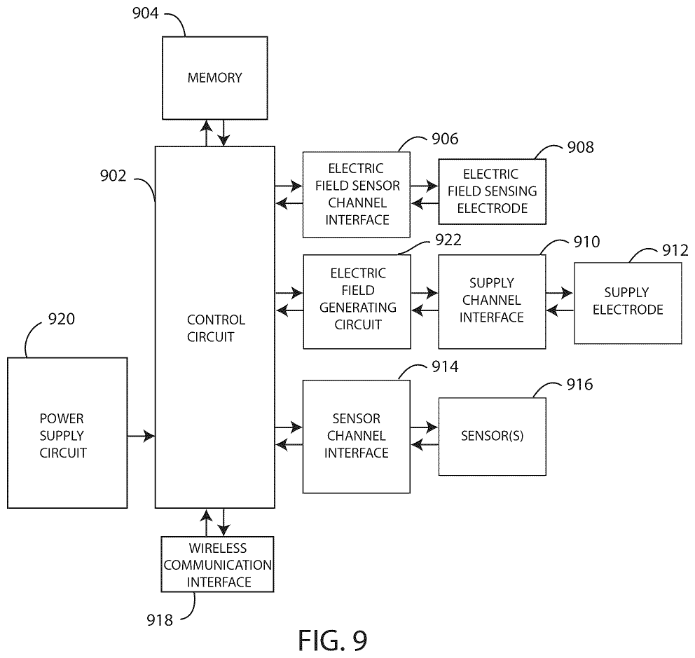

Elements of various embodiments of the medical devices described herein are shown in FIG. 9. However, it will be appreciated that some embodiments can include additional elements beyond those shown in FIG. 9. In addition, some embodiments may lack some elements shown in FIG. 9. The medical devices as embodied herein can gather information through one or more sensing channels and can output information through one or more field generating channels. A microprocessor 902 can communicate with a memory 904 via a bidirectional data bus. The memory 904 can include read only memory (ROM) or random-access memory (RAM) for program storage and RAM for data storage. The microprocessor 902 can also be connected to a wireless communication interface 918 for communicating with external devices such as a programmer, a home-based unit and/or a mobile unit (e.g., a cellular phone, personal computer, smart phone, tablet computer, and the like) or directly to the cloud or another communication network as facilitated by a cellular or other data communication network. The medical device can include a power supply circuit 920. In some embodiments, the medical device can include an inductive energy receiver coil interface (not shown) communicatively coupled or attached thereto to facilitate transcutaneous recharging of the medical device.

The medical device can include one or more electric field sensing electrodes 908 and one or more electric field sensor channel interfaces 906 that can communicate with a port of microprocessor 902. The medical device can also include one or more electric field generating circuits 922, one or more supply electrodes 912, and one or more supply channel interfaces 910 that can communicate with a port of microprocessor 902. The medical device can also include one or more sensors 916 (such as temperature sensors) and one or more sensor channel interfaces 914 that can communicate with a port of microprocessor 902. The channel interfaces 906, 910, and 914 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like.

In various embodiments, the electric field generating circuit 922, the supply channel interface 910, or various other portions of the system along a conductive path for the electrodes can include components including, but not limited to, variable resistors, variable capacitors, and/or field effect transistors, and the like to change the impedance thereof in order to facilitate changes in current distribution as described herein.

In some embodiments, the electric field generating circuit 922, the supply channel interface 910, or various other portions of the system along a conductive path for the electrodes can include one or more current monitors or current sensors. Current sensing can be performed in various ways. In some embodiments, components such as shunt resistors, current transformers and Rogowski coils, and/or magnetic-field based transducers can be used in order to sense current along a conductive path for electrodes herein.

In some embodiments, one or more physiological sensors can also be included herein. In some embodiments, the physiological sensors can include sensors that monitor temperature, blood flow, blood pressure, and the like. In some embodiments, the respiration sensors can include sensors that monitor respiration rate, respiration peak amplitude, and the like. In some embodiments, the chemical sensors can measure the quantity of an analyte present in a treatment area about the sensor, including but not limited to analytes such as of blood urea nitrogen, creatinine, fibrin, fibrinogen, immunoglobulins, deoxyribonucleic acids, ribonucleic acids, potassium, sodium, chloride, calcium, magnesium, lithium, hydronium, hydrogen phosphate, bicarbonate, and the like. However, many other analytes are also contemplated herein. Exemplary chemical/analyte sensors are disclosed in commonly owned U.S. Pat. No. 7,809,441 to Kane et al., and which is hereby incorporated by reference in its entirety.

Although the temperature sensors 916 are shown as part of a medical device in FIG. 9, it is realized that in some embodiments one or more of the sensors could be physically separate from the medical device. In various embodiments, one or more of the can be within another implanted medical device communicatively coupled to a medical device via wireless communication interface 918. In yet other embodiments, one or more of the sensors can be external to the body and coupled to a medical device via wireless communication interface 918.

### Electric Field Therapy Parameters

In some embodiments, medical devices herein can generate one or more electric fields at frequencies selected from a range of between 10 kHz to 1 MHz. In some embodiments, the one or more electric fields can be effective to prevent and/or disrupt cellular mitosis in a cell. In some embodiments, the one or more electric fields can be effective to prevent and/or disrupt cellular mitosis in a cell, but not cause tissue ablation. In some embodiments, the system can be configured to deliver an electric field at one or more frequencies selected from a range of within 300 kHz to 500 kHz. In some embodiments, the system can be configured to deliver an electric field at one or more frequencies selected from a range of within 100 kHz to 300 kHz. In some embodiments, the system can be configured to periodically deliver an electric field using one or more frequencies greater than 10 kHz.

A desired electric field strength can be achieved by delivering an electric current between two electrodes. The specific current and voltage at which the electric field is delivered can vary and can be adjusted to achieve the desired electric field strength at the site of the tissue to be treated. In some embodiments, the system can be configured to deliver an electric field using currents ranging from 1 mAmp to 1000 mAmp to the site of a cancerous tumor. In some embodiments, the system can be configured to deliver an electric field using currents ranging from 20 mAmp to 500 mAmp to the site of a cancerous tumor. In some embodiments, the system can be configured to deliver an electric field using currents ranging from 30 mAmp to 300 mAmp to the site of a cancerous tumor.

In some embodiments, the system can be configured to deliver an electric field using currents including 1 mAmp, 2 mAmp, 3 mAmp, 4 mAmp, 5 mAmp, 6 mAmp, 7 mAmp, 8 mAmp, 9 mAmp, 10 mAmp, 15 mAmp, 20 mAmp, 25 mAmp, 30 mAmp, 35 mAmp, 40 mAmp, 45 mAmp, 50 mAmp, 60 mAmp, 70 mAmp, 80 mAmp, 90 mAmp, 300 mAmp, 125 mAmp, 150 mAmp, 175 mAmp, 400 mAmp, 225 mAmp, 250 mAmp, 275 mAmp, 300 mAmp, 325 mAmp, 350 mAmp, 375 mAmp, 400 mAmp, 425 mAmp, 450 mAmp, 475 mAmp, 500 mAmp, 525 mAmp, 550 mAmp, 575 mAmp, 600 mAmp, 625 mAmp, 650 mAmp, 675 mAmp, 700 mAmp, 725 mAmp, 750 mAmp, 775 mAmp, 800 mAmp, 825 mAmp, 850 mAmp, 875 mAmp, 900 mAmp, 925 mAmp, 950 mAmp, 975 mAmp, or 1000 mAmp. It will be appreciated that the system can be configured to deliver an electric field at a current falling within a range, wherein any of the forgoing currents can serve as the lower or upper bound of the range, provided that the lower bound of the range is a value less than the upper bound of the range.

In some embodiments, the system can be configured to deliver an electric field using voltages ranging from 1 Vrms to 50 Vrms to the site of a cancerous tumor. In some embodiments, the system can be configured to deliver an electric field using voltages ranging from 5 Vrms to 30 Vrms to the site of a cancerous tumor. In some embodiments, the system can be configured to deliver an electric field using voltages ranging from 10 Vrms to 20 Vrms to the site of a cancerous tumor.

In some embodiments, the system can be configured to deliver an electric field using one or more voltages including 1 Vrms, 2 Vrms, 3 Vrms, 4 Vrms, 5 Vrms, 6 Vrms, 7 Vrms, 8 Vrms, 9 Vrms, 10 Vrms, 15 Vrms, 20 Vrms, 25 Vrms, 30 Vrms, 35 Vrms, 40 Vrms, 45 Vrms, or 50 Vrms. It will be appreciated that the system can be configured to deliver an electric field at a voltage falling within a range, wherein any of the forgoing voltages can serve as the lower or upper bound of the range, provided that the lower bound of the range is a value less than the upper bound of the range.

In some embodiments, the system can be configured to deliver an electric field using one or more frequencies including 10 kHz, 20 kHz, 30 kHz, 40 kHz, 50 kHz, 60 kHz, 70 kHz, 80 kHz, 90 kHz, 300 kHz, 125 kHz, 150 kHz, 175 kHz, 400 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 MHz. It will be appreciated that the system can be configured to deliver an electric field using a frequency falling within a range, wherein any of the foregoing frequencies can serve as the upper or lower bound of the range, provided that the upper bound is greater than the lower bound.

In some embodiments, the system can be configured to generate one or more applied electric field strengths selected from a range of within 0.25 V/cm to 1000 V/cm, or 0.25 V/cm to 500 V/cm, or 0.25 V/cm to 100 V/cm, or 0.25 V/cm to 50 V/cm. In some embodiments, the system can be configured to generate one or more applied electric field with strengths of greater than 3 V/cm. In some embodiments, the system can be configured to generate one or more applied electric field strengths selected from a range of within 1 V/cm to 10 V/cm. In some embodiments, the system can be configured to generate one or more applied electric field strengths selected from a range of within 3 V/cm to 5 V/cm.

In other embodiments, the system can be configured to deliver one or more applied electric field strengths including 0.25 V/cm, 0.5 V/cm, 0.75 V/cm, 1.0 V/cm, 2.0 V/cm, 3.0 V/cm, 5.0 V/cm, 6.0 V/cm, 7.0 V/cm, 8.0 V/cm, 9.0 V/cm, 10.0 V/cm, 20.0 V/cm, 30.0 V/cm, 40.0 V/cm, 50.0 V/cm, 60.0 V/cm, 70.0 V/cm, 80.0 V/cm, 90.0 V/cm, 300.0 V/cm, 125.0 V/cm, 150.0 V/cm, 175.0 V/cm, 400.0 V/cm, 225.0 V/cm, 250.0 V/cm, 275.0 V/cm, 300.0 V/cm, 325.0 V/cm, 350.0 V/cm, 375.0 V/cm, 400.0 V/cm, 425.0 V/cm, 450.0 V/cm, 475.0 V/cm, 500.0 V/cm, 600.0 V/cm, 700.0 V/cm, 800.0 V/cm, 900.0 V/cm, 1000.0 V/cm. It will be appreciated that the system can generate an electric field having a field strength at a treatment site falling within a range, wherein any of the foregoing field strengths can serve as the upper or lower bound of the range, provided that the upper bound is greater than the lower bound.

In some embodiments, an electric field can be applied to the site of a cancerous tumor or tumor resection at a specific frequency or constant frequency range.

In some embodiments, the electric field can be modulated in response to a patient's measured reference or core body temperature and/or a set period of time elapsing. For example, if the patient's reference or core body temperature is higher than a threshold level, the therapy parameters can be modulated to reduce the heat output of the system inside the body. If the patient's reference or core body temperature is higher than a threshold level, the electric field strength can be decreased by 5%, 10%, 15%, 20%, 25%, 30%, 35%, 40%, 45%, 50%, 55%, 60%, 65%, 70%, 75%, 80%, 85%, 90%, 95%, or 100% (e.g., turned off) or can be decreased by an amount falling within a range between any of the foregoing. If the patient's reference or core body temperature is higher than a threshold level, the electric field strength can be decreased by from 5% to 100%, or between 5% and 95%. It will be appreciated that other parameters can also be modulated in order to reduce the amount of heat generated by the system including, for example, current, voltage, and/or frequency.

Alternatively, if the reference or core body temperature is lower than a threshold level, then to maximize exposure to therapeutic electrical fields the electric field strength can be increased. In some embodiments, the electric field strength can be increased by 5%, 10%, 15%, 20%, 25%, 50%, 75%, 100%, 200%, 300%, 500%, 1000% or more, or by an amount falling within a range between any of the foregoing. It will be appreciated that other parameters can also be modulated in order to increase the intensity of the electrical field therapy provided by the system including, for example, current, voltage, and/or frequency.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An implantable system for cancer treatment comprising:
a plurality of implantable stimulation lead;
a plurality of electric field therapy electrodes;
a therapy output circuit, wherein the therapy output circuit is configured to generate an electrical current for the plurality of electric field therapy electrodes to create one or more electric fields; and
control circuitry;
wherein the control circuitry causes the therapy output circuit to generate the one or more electric fields at frequencies selected from a range of between 10 kHz to 1 MHz within a bodily tissue, wherein the one or more electric fields are effective to prevent and/or disrupt cellular mitosis in a cell;
wherein the control circuitry is configured to cause the electrical current to be distributed asymmetrically between working electrodes and counter electrodes amongst the plurality of electric field therapy electrodes; and
wherein at least some of the plurality of electric field therapy electrodes are disposed on the plurality of implantable stimulation lead and in electrical communication with the therapy output circuit.

2. The implantable system of claim 1, wherein the control circuitry is configured to cause the electrical current to be distributed asymmetrically between working electrodes and counter electrodes amongst the plurality of electric field therapy electrodes by pairing unequal numbers of working electrodes with counter electrodes.

3. The implantable system of claim 1, wherein the control circuitry is configured to cause the electrical current to be distributed asymmetrically between working electrodes and counter electrodes amongst the plurality of electric field therapy electrodes by duty cycling different working electrodes with a particular counter electrode and/or different counter electrodes with a particular working electrode.

4. The implantable system of claim 1, further comprising a metallic housing;
wherein the therapy output circuit is disposed within the metallic housing.

5. The implantable system of claim 4, wherein the metallic housing is configured to serve as a working electrode or a counter electrode.

6. The implantable system of claim 1, further comprising a plurality of current monitors, wherein the plurality of current monitors are configured to monitor current to the plurality of electric field therapy electrodes.

7. The implantable system of claim 1, further comprising a plurality of variable selective resistors, wherein the plurality of variable selective resistors are configured to change impedance for the plurality of electric field therapy electrodes.

8. A method of providing cellular mitosis disrupting cancer therapy comprising:
generating an electrical current for a plurality of electric field therapy electrodes to create one or more electric fields; and
causing the electrical current to be distributed asymmetrically between working electrodes and counter electrodes amongst the plurality of electric field therapy electrodes.

9. The method of claim 8, further comprising causing the electrical current to be distributed asymmetrically between working electrodes and counter electrodes amongst the plurality of electric field therapy electrodes by pairing unequal numbers of working electrodes with counter electrodes.

10. The method of claim 8, further comprising causing the electrical current to be distributed asymmetrically between working electrodes and counter electrodes amongst the plurality of electric field therapy electrodes by duty cycling different working electrodes with a particular counter electrode and/or different counter electrodes with a particular working electrode.

11. The method of claim 8, further comprising monitoring current to the plurality of electric field therapy electrodes.

12. The method of claim 8, further comprising changing impedance for the plurality of electric field therapy electrodes.

13. An implantable system for cancer treatment comprising:
a plurality of implantable stimulation lead;
a plurality of electric field therapy electrodes;
a therapy output circuit, wherein the therapy output circuit is configured to generate an electrical current for the plurality of electric field therapy electrodes to create one or more electric fields; and
control circuitry;
wherein the control circuitry causes the therapy output circuit to generate the one or more electric fields at frequencies selected from a range of between 10 kHz to 1 MHz within a bodily tissue, wherein the one or more electric fields are effective to prevent and/or disrupt cellular mitosis in a cell;
wherein the control circuitry is configured to cause the electrical current to be distributed between working electrodes and counter electrodes amongst the plurality of electric field therapy electrodes such that some electrodes have a higher operating temperature during therapy than other electrodes; and
wherein at least some of the plurality of electric field therapy electrodes are disposed on the plurality of implantable stimulation lead and in electrical communication with the therapy output circuit.

14. The implantable system of claim 13, wherein the control circuitry is configured to cause the electrical current to be distributed between working electrodes and counter electrodes amongst the plurality of electric field therapy electrodes such that some electrodes have a higher operating temperature during therapy than other electrodes by distributing current amongst the electrodes asymmetrically.

15. The implantable system of claim 13, wherein the control circuitry is configured to cause the electrical current to be distributed asymmetrically between working electrodes and counter electrodes amongst the plurality of electric field therapy electrodes by pairing unequal numbers of working electrodes with counter electrodes.

16. The implantable system of claim 13, wherein the control circuitry is configured to cause the electrical current to be distributed asymmetrically between working electrodes and counter electrodes amongst the plurality of electric field therapy electrodes by duty cycling different working electrodes with a particular counter electrode and/or different counter electrodes with a particular working electrode.

17. The implantable system of claim 13, further comprising a metallic housing, wherein the therapy output circuit is disposed within the metallic housing.

18. The implantable system of claim 17, wherein the metallic housing is configured to serve as a working electrode or a counter electrode.

19. The implantable system of claim 13, further comprising a plurality of current monitors, wherein the plurality of current monitors are configured to monitor current to the plurality of electric field therapy electrodes.

20. The implantable system of claim 13, further comprising a plurality of variable selective resistors, wherein the plurality of variable selective resistors are configured to change impedance for the plurality of electric field therapy electrodes.
